(19) United States
(12) Patent Application Publication
(10) Pub. No.: US 2009/0149436 A1
(43) Pub. Date: Jun. 11, 2009

(54) CASPASE INHIBITOR

(75) Inventors: Hirohisa Nakada, Tokyo (JP); Takuya Maemoto, Tokyo (JP); Yuko Muramatsu, Tokyo (JP)

Correspondence Address: OBLON, SPIVAK, MCCLELLAND MAIER & NEUSTADT, P.C., 1940 DUKE STREET, ALEXANDRIA, VA, 22314, US

(73) Assignee: Astellas Pharma Inc., Chuo-ku (JP)

(21) Appl. No.: 11/719,468

(22) PCT Filed: Nov. 16, 2005
(86) PCT No.: PCT/JP05/21396

§ 371 Date: May 16, 2007

(30) Foreign Application Priority Data
Nov. 16, 2004 (JP) 2004-331331

(51) Int. Cl. A61K 31/555; C07D 487/22; A61P 25/28; A61P 25/16

(52) U.S. Cl. 514/185; 540/145

(57) ABSTRACT

The present invention provides a caspase inhibitor containing a cobalt porphyrin complex compound or cobalt choline complex compound as an active ingredient. The caspase inhibitor of the present invention can strongly and specifically inhibit the caspase activity. In addition, the caspase inhibitor/cobalt porphyrin complex compound or cobalt choline complex compound of the present invention is useful as a pharmaceutical agent for the prophylaxis or treatment of various diseases relating to apoptosis.

### TECHNICAL FIELD

The present invention relates to a caspase inhibitor comprising a compound that strongly and specifically inhibits caspase activity as an active ingredient.

### BACKGROUND ART

Caspases are also called ICE family proteases, and refer to a group of cysteine proteases that show function in the implementation of apoptosis, and processing of cytokines (interleukin-1β (IL-1β) interferon-γ-inducer (IGIF and the like) important for inflammation reactions. To the presents many caspase homologs have been obtained from mammals, and at least 12 kinds are known. Caspases are divided into three groups I-III based on the substrate specificity. Group I is represented by caspase 1, which is involved in the processing of cytokine and apoptosis, group II is represented by caspase 3, which is involved in the implementation of apoptosis, and group III is represented by caspase 8, which is located at the upstream of a proteolytic cascade of caspase and involved in the apoptosis signal transduction.

Caspase inhibitors are being intensively screened for worldwide as pharmaceutical agents capable of inhibiting cytopathy in various pathologies such as neuropathy (e.g., cerebral ischemia, Alzheimer's disease), hepatitis, diabetic organopathy, genetic disease and the like. Particularly, design and synthesis of inhibitors having high enzyme specificity based on differences in the active center structures of the subtype enzymes has been actively performed along with the development of stereochemistry. However, a caspase inhibitor successfully subjected to clinical trials has not been produced to date.

### DISCLOSURE OF THE INVENTION

In view of the above-mentioned situation, the present inventors have performed random screening over a broad range for caspase inhibitors and found a group of compounds that strongly and specifically inhibit the caspase activity. More particularly, the present inventors have found that various complex compounds wherein a choline ring structure or porphyrin ring structure is coordinated to cobalt strongly and specifically inhibit the caspase activity, which resulted in the completion of the present invention.

Accordingly, the present invention provides the following.

[1] A caspase inhibitor comprising a cobalt porphyrin complex compound or a cobalt choline complex compound as an active ingredient.
[2] The caspase inhibitor of the above-mentioned [1], wherein the aforementioned cobalt porphyrin complex compound or cobalt choline complex compound is a cobyrinic acid derivative.
[3] The caspase inhibitor of the above-mentioned [2], wherein the aforementioned cobyrinic acid derivative is a cobyric acid derivative.
[4] The caspase inhibitor of the above-mentioned [3], wherein the aforementioned cobyric acid derivative is dicyanocobinamide.
[5] The caspase inhibitor of the above-mentioned [1], wherein the aforementioned caspase is caspase 3.
[6] A pharmaceutical composition for the prophylaxis or treatment of an apoptosis-associated diseases which comprises a cobalt porphyrin complex compound or cobalt choline complex compound in an amount effective for the treatment.
[7] The pharmaceutical composition of the above-mentioned [6], wherein the aforementioned cobalt porphyrin complex compound or cobalt choline complex compound is a cobyrinic acid derivative.
[8] The pharmaceutical composition of the above-mentioned [7], wherein the aforementioned cobyrinic acid derivative is a cobyric acid derivative.

[9] The pharmaceutical composition of the above-mentioned [8], wherein the aforementioned cobyric acid derivative is dicyanocobinamide.

[10] The pharmaceutical composition of the above-mentioned [6], wherein the aforementioned apoptosis-associated disease is Alzheimer, Parkinson's syndrome or amyotropic lateral sclerosis.
[11] A method for inhibiting caspase, comprising applying a cobalt porphyrin complex compound or cobalt choline complex compound.
[12] The method of the above-mentioned [11], wherein the aforementioned cobalt porphyrin complex compound or cobalt choline complex compound is a cobyrinic acid derivative.
[13] The method of the above-mentioned [12] wherein the aforementioned cobyrinic acid derivative is a cobyric acid derivative.
[14] The method of the above-mentioned [13], wherein the aforementioned cobyric acid derivative is dicyanocobinamide.
[15] The method of the above-mentioned [11], wherein the aforementioned caspase is caspase 3.
[16] A method for the prophylaxis or treatment of an apoptosis-associated diseases comprising a step for administering a cobalt porphyrin complex compound or cobalt choline complex compound in an amount effective for the treatment.
[17] The method of the above-mentioned [16] wherein the aforementioned cobalt porphyrin complex compound or cobalt choline complex compound is a cobyrinic acid derivative.
[18] The method of the above-mentioned [17], wherein the aforementioned cobyrinic acid derivative is a cobyric acid derivative.
[19] The method of the above-mentioned [18] wherein the aforementioned cobyric acid derivative is dicyanocobinamide.
[20] The method of the above-mentioned [16], wherein the aforementioned apoptosis-associated disease is Alzheimer, Parkinson's syndrome or amyotropic lateral sclerosis.
[21] Use of a cobalt porphyrin complex compound or cobalt choline complex compound for the production of a caspase inhibitor.
[22] The use of the above-mentioned [21] wherein the aforementioned cobalt porphyrin complex compound or cobalt choline complex compound is a cobyrinic acid derivative.
[23] The use of the above-mentioned [22], wherein the aforementioned cobyrinic acid derivative is a cobyric acid derivative.
[24] The use of the above-mentioned [23], wherein the aforementioned cobyric acid derivative is dicyanocobinamide.
[25] The use of the above-mentioned [21], wherein the aforementioned caspase is caspase 3.
[26] Use of a cobalt porphyrin complex compound or cobalt choline complex compound for the production of a pharmaceutical agent for the prophylaxis or treatment of an apoptosis-associated disease.
[27] The use of the above-mentioned [26], wherein the aforementioned cobalt porphyrin complex compound or cobalt choline complex compound is a cobyrinic acid derivative.
[28] The use of the above-mentioned [27], wherein the aforementioned cobyrinic acid derivative is a cobyric acid derivative.
[29] The use of the above-mentioned [28], wherein the aforementioned cobyric acid derivative is dicyanocobinamide.
[30] The use of the above-mentioned [26], wherein the aforementioned apoptosis-associated disease is Alzheimer, Parkinson's syndrome or amyotropic lateral sclerosis.
[31] A commercial package comprising the caspase inhibitor of the above-mentioned [1] and a written matter associated therewith, the written matter stating that the caspase inhibitor can or should be used for the prophylaxis or treatment of an apoptosis-associated disease.
[32] The commercial package of the above-mentioned [31], wherein the aforementioned anoptosis-associated disease is Alzheimer, Parkinson's syndrome or amyotropic lateral sclerosis.

### BRIEF DESCRIPTION OF THE DRAWINGS

FIG. 1 shows the data of the activity inhibitory action by a cobalt porphyrin complex compound and a cobalt choline complex compound on human recombinant caspase 3.

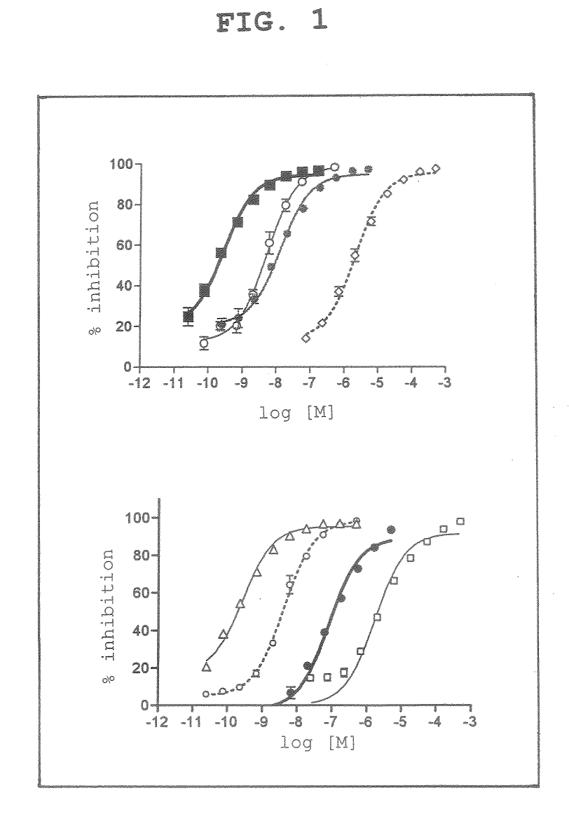

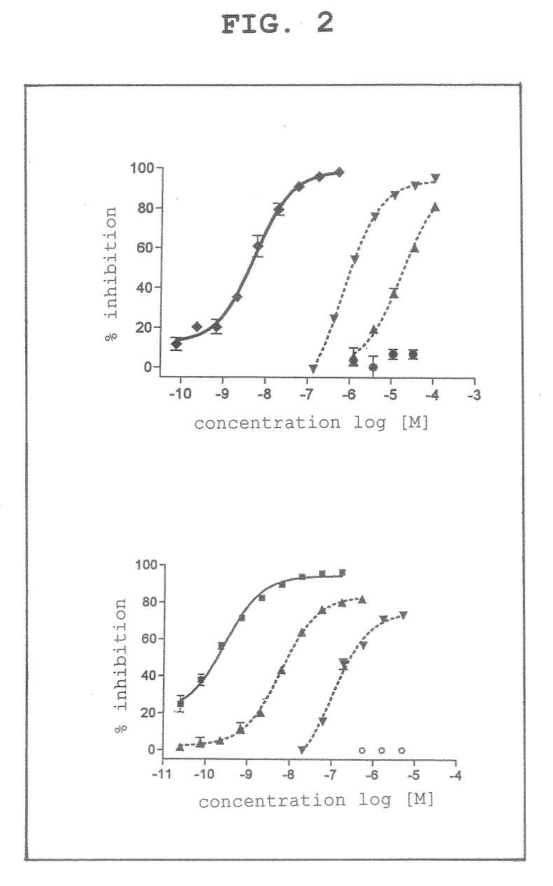

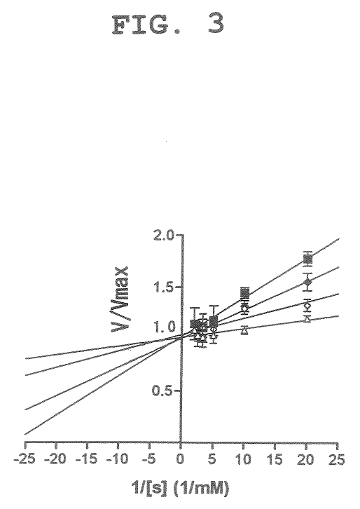

In the upper panel of FIG. 1 (inhibitory activity against human recombinant caspase 3, Recombinant Caspase 3 Assay), each symbol means the following.

| | |
|---|---|
| ▪ dicyanocobinamide (Cobinamide) | IC50 = 0.28 nM |
| cobalt protoporphyrin (CoPP) | IC50 = 13.1 nM |
| ◯ DEVD | IC50 = 5.19 nM |
| ⋄ cyanocobalamin (VB12) | IC50 = 2.10 μM |

In the lower panel of FIG. 1 (inhibitory activity against human recombinant caspase 3, Recombinant Caspase 3 Assay), each symbol means the following.

| | |
|---|---|
| □ cyanocobalamin (VB12) | IC50 = 1.76 μM |
| Δ dicyanocobinamide (Cobinamide) | IC50 = 0.142 nM |
| ◯ DEVD | IC50 = 3.54 nM |
| cyanoimidazolylcobamide (ImdCobamd) | IC50 = 91.2 nM |

DEVD means acetyl-L-aspartyl-L-glutamyl-L-valyl-7-aspart-1-al.

FIG. 2 shows the specificity of the inhibitory activity of dicyanocobinamide and DEVD as a comparative test against human recombinant caspase 3.

To be specific, the upper panel of FIG. 2 shows the inhibitory activity of DEVD (Enzyme Specificity; DEVD-CHO) against each protease, wherein each symbol means the following.

| | |
|---|---|
| ♦ caspase (Caspase) | IC50 = 5.19 nM |
| ▴ cathepsin L (Cathespin L) | IC50 = 17.4 μM |
| ▾cathepsin B (Cathespin B) | IC50 = 672 nM |
| trypsin (Trypsin) | IC50 > 100 μM |

The lower panel of FIG. 2 shows the inhibitory activity of dicyanocobinamide (Enzyme Specificity; Cobinamide) against each protease, wherein each symbol means the following.

| | |
|---|---|
| ▪ caspase (Caspase) | IC50 = 0.28 nM |
| ▴cathepsin L (Cathepsin L) | IC50 = 6.12 nM |
| ▾ cathepsin B (Cathepsin B) | IC50 = 102 nM |
| ◯ trypsin (Trypsin) | IC50 > 5 μM |

FIG. 3 shows the inhibitory mode (Lineweaver-Burk plot) of dicyanocobinamide (Cobinamide) against human recombinant caspase 3.

In FIG. 3, each symbol means the following.

Δ:2 nM

⋄:4 nM

♦:6 nm

▪:8 nM

### DETAILED DESCRIPTION OF THE INVENTION

The present invention is described in detail in the following.

### BEST MODE FOR EMBODYING THE INVENTION

The “cobalt porphyrin complex compound” and “cobalt choline complex compound” to be used for the caspase inhibitor of the present invention are each a compound having a complex structure wherein a porphyrin ring structure or a choline ring structure is coordinated to a cobalt atom, as shown by the following A and B:

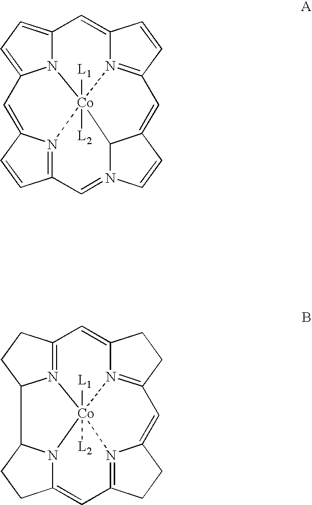

wherein L₁ and L₂ are each independently, any ligand relative to cobalt, which may or may not be present, and when it is present, each independently, for example, H₂O, a cyano group, a hydroxyl group, a methyl group, an imidazolyl group or an adenosyl group.

A more preferable “cobalt porphyrin complex compound” or “cobalt choline complex compound” to be used for the caspase inhibitor of the present invention is a “cobyrinic acid derivative”. In the present specification, a “cobyrinic acid derivative” refers to a compound having a structure represented by the following formula:

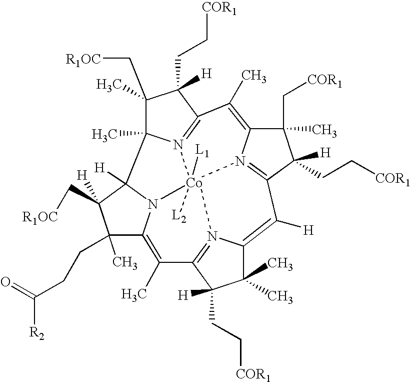

wherein each R₁ is independently, for example, a hydroxyl group, an amino group or a lower alkoxy group having 1 to 6 carbon atoms (e.g., methoxy, ethoxy, propoxy, isopropoxy, butoxy, isobutoxy, sec-butoxy, tert-butoxy, pentyloxy, isopentyloxy, neopentyloxy, hexyloxy etc.), R₂ is, for example, a hydroxyl group, an amino group, an optionally substituted alkylamino group or an optionally substituted or esterified hydroxyalkylamino group, and L₁ and L₂ are each independently any ligand relative to cobalt, which may or may not be present, and when it is present, each independently, for example, H₂O, a cyano group, a hydroxyl group, a methyl group, an imidazolyl group or an adenosyl group.

The “alkylamino group” of the above-mentioned “optionally substituted alkylamino group” is an amino group having 1 or 2 lower alkyl groups having 1 to 6 carbon atoms (e.g., methyl, ethyl, propyl, isopropyl, butyl, isobutyl, sec-butyl, tert-butyl, pentyl, isopentyl, neopentyl, hexyl and the like) and, for example, methylamino, ethylamino, propylamino, isopropylamino, butylamino, isobutylamino, sec-butylamino, tert-butylamino, pentylamino, isopentylamino, neopentylamino, hexylamino and the like can be mentioned. The substituent and the number thereof that the alkylamino group may have are not particularly limited.

The “hydroxyalkylamino group” of the above-mentioned “optionally substituted or esterified hydroxyalkylamino group” means the above-mentioned “alkylamino group” having a hydroxyl group at a substitutable position and, for example, hydroxymethylamino, 1-hydroxyethylamino, 2-hydroxyethylamino, 1-hydroxypropylamino, 2-hydroxypropylamino, 3-hydroxypropylamino and the like can be mentioned. The substituent and the number thereof that the hydroxyalkylamino group may have are not particularly limited. The hydroxy moiety of the hydroxyalkylamino group may further form an ester with α-D-ribofuranose 3-phosphoric acid, imidazolyl-α-D-ribofuranose 3-phosphoric acid, 5,6-dimethylbenzimidazolyl-α-D-ribofuranose 3-phosphoric acid and the like.

Examples of these cobyrinic acid derivatives include, but not limited to, cobinamide, cobamide, cobyrinamide, cobyrinic acid, cobyric acid, cobinic acid, cobamic acid and cobalamin, and cobyrinic acid derivatives having a ligand to the cobalt atom of these compounds, such as dicyanocobinamide, adenosylcobyrinamide, imidazolylcobalamin, cobalt protoporphyrin, cyanoimidazolylcobamide, cyanocobalamin and the like.

More preferably, the “cobyrinic acid derivative” to be used as the caspase inhibitor of the present invention is a “cobyric acid derivative”, wherein all R₁ in the above-mentioned formula are amino groups. Examples of the cobyric acid derivatives include, but not limited to, cobyric acid derivatives such as cobinamide, cobamide, cobyrinamide, cobyric acid, cobalamin and the like.

Still more preferably, the “cobyric acid derivative” to be used as the caspase inhibitor of the present invention is dicyanocobinamide having the following formula:

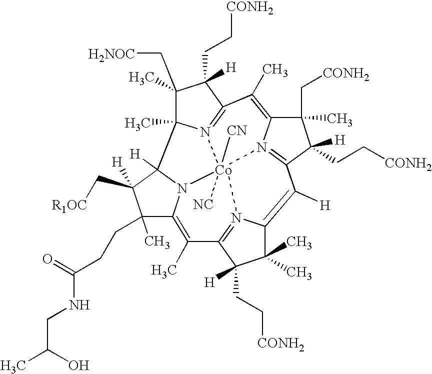

The cobalt porphyrin complex compound and cobalt choline complex compound to be used for the caspase inhibitor of the present invention is available from various supply sources known in the fields or easily produced by those of ordinary skill in the art using various chemical synthesis techniques known in the field. The synthesis methods of the cobalt porphyrin complex compound or cobalt choline complex compound known in the field are disclosed, for example, in Y. Murakami et al., Chem. Lett. 469 (1988), Bull. Chem. Soc. Jpn. 60, 311 (1987), B Gruening et al., Helv. Chim. Acta 68, 1771 (1985), Y. Murakami et al. Chem Lett. 477 (1985), B. Teeiger et al., Chimia 45, 32 (1991), Review: S. Fukui, S. Shimizu, “Antibiotics, Vitamins and Hormones” (F. Korte, M. Cotoo eds. 101 (1977, George Thieme Stuttgart and the like, and available from supply sources such as Fluka, Sigma-Aldrich, Cosmo Bio and the like.

The cobalt porphyrin complex compound and cobalt choline complex compound to be used for the caspase inhibitor of the present invention encompasses pharmaceutically acceptable salts thereof and metal salts (e.g., alkali metal salt such as sodium salt potassium salt and the like; alkaline earth metal salt such as calcium salt, magnesium salt, barium salt and the like etc.), salts with inorganic base (e.g., ammonium salt etc.), salts with organic base (e.g. trimethylamine, triethylamine, pyridine, picoline, 2,6-lutidine, ethanolamine, diethanolamine, triethanolamine, cyclohexylamine, dicyclohexylamlne, N,N′-dibenzylethylenediamine etc.), addition salts of inorganic acid (e.g., hydrochloric acid, hydrobromic acid, nitric acid, sulfuric acid, phosphoric acid etc.), addition salts of organic acid (e.g., formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid etc.), salts with basic amino acid (e.g., arginine, lysine, ornithine etc.) or acidic amino acid (e.g., aspartic acid, glutamic acid etc. and the like.

As the caspase that can be inhibited by the caspase inhibitor/cobalt porphyrin complex compound or cobalt choline complex compound of the present invention, various caspases known in the field can be mentioned, and preferably, caspase 3 can be inhibited.

Examples of the apoptosis-associated diseases that can be prevented and/or treated by the caspase inhibitor/cobalt porphyrin complex compound or cobalt choline complex compound of the present invention include, but not limited to, encephalopathy (e.g., cerebrovascular accident dementia, multiple cerebral microinfarcts, cerebrovascular thrombosis, cerebral infarction, cerebral hemorrhage and the like), reurodegenerative diseases (Alzheimer, Parkinson's syndrome, amyotropic lateral sclerosis and the like), cardiac infarction, myocarditis, viral hepatitis alcoholic hepatitis cirrhosis insulin-dependent diabetes, ischemic enteritis, lung disease, systemic or local autoimmune diseases, systemic diffuse erythema, dermatomyositis, chronic articular rheumatism, human immunodeficiency virus (HIV) immunodeficiency syndrome, transplant rejection, cancer and other proliferative diseases and the like.

When the caspase inhibitor/cobalt porphyrin complex compound or cobalt choline complex compound of the present invention is used as a therapeutic agent inclusive of prophylactic agent and/or therapeutic agent), it is also desirably used as a part of a prescribed preparation. Such an “apoptosis-associated disease therapeutic agent” is contained as a mixture with at least one, or several suitable organic or inorganic carriers or excipients, or other pharmacological therapeutic agent can be used, for example, as a solid, semisolid or liquid pharmaceutical preparation. The active ingredient is mixed, for example, with a pharmaceutically conventional non-toxic carriers and can be processed into granule, tablet, pellet, troche, capsule, suppository, cream, ointment, aerosol, inhalation powder, liquid such as injection liquid, emulsion, suspension and the like; agent for oral intale; eye drop; or any form suitable for use. Where necessary, auxiliary agents such as stabilizer, thickener, wetting agent, curing agent, coloring agent and the like; flavoring or buffer; any other conventional additive can be added to the above-mentioned preparation.

The subject of administration of the caspase inhibitor/cobalt porphyrin complex compound or cobalt choline complex compound of the present invention is not particularly limited and, for example, mammal (e.g., mouse, rat, hamster, rabbit, cat, dog, bovines sheep, monkey, human etc.) and the like can be mentioned.

The effective amount of the caspase inhibitor/cobalt porphyrin complex compound or cobalt choline complex compound of the present invention to be administered for the prophylaxis and/or treatment of apoptosis-associated diseases varies depending on the age and condition of the target/patient, and is also dependent on the type of the prescription for which the therapeutic agent is administered and the administration mode thereof, and the stage of the disease or administration frequency. For example, the active ingredient is generally administered within the range of 0.00001 mg-1000 mg, preferably, 0.001 mg-100 mg, per 1 kg/body weight/adult for one day. In consideration of various conditions, however, the dose can be changed, where necessary to a smaller or higher amount than the above-mentioned ranges.

In one aspect, the present invention provides a caspase inhibitor comprising a cobalt porphyrin complex compound or cobalt choline complex compound as an active ingredient.

In one embodiment, the aforementioned cobalt porphyrin complex compound or cobalt choline complex compound is a cobyrinic acid derivative.

In a preferable embodiment, the aforementioned cobyrinic acid derivative is a cobyric acid derivative.

In a more preferable embodiment the aforementioned cobyric acid derivative is dicyanocobinamide.

In one embodiment, the aforementioned caspase is caspase 3.

The above-mentioned caspase inhibitor of the present invention is considered to be useful as a pharmaceutical agent for the prophylaxis or treatment of various diseases relating to apoptosis.

Therefore, in a different aspect, the present invention provides a pharmaceutical composition for the prophylaxis or treatment of apoptosis-associated diseases, which comprises the aforementioned caspase inhibitor in an amount effective for the treatment. In another aspect, the present invention provides a method for the prophylaxis or treatment of apoptosis-associated diseases which includes a step of administering the aforementioned caspase inhibitor in an amount effective for the treatment.

In a different aspect, the present invention provides use of the aforementioned caspase inhibitor for the production of a pharmaceutical agent for the prophylaxis or treatment of apoptosis-associated diseases.

The present invention is explained in more detail in the following by referring to Examples, which are not to be construed as limitative.

### EXAMPLES

### Material and Method

### (1. Cobalt Porphyrin Complex Compound and Cobalt Choline Complex Compound)

The cobalt porphyrin complex compound and cobalt choline complex compound used for the following experiments were as follows: cobalt protoporphyrin (Cosmo Bio catalog No.: 430076M025), dicyanocobinamide (Fluka catalog No.: 36612), cyanocobalamin (Fluka catalog No.: 95190) and cyanoimidazolylcobamide (supplied by Dr. Erhard Stupperich (Abteilung Angewandte Mikrobilogie der Universitaet Ulm) (available by fermentation method)).

### (2. Assay Method of Inhibitory Activity Against Human Recombinant Caspase 3)

A reaction buffer containing 10% sucrose, 0.1% CHAPS, 2 mM dithiothreitol in 25 mM HEPES (pH 7.5) is prepared. A pharmaceutical agent-diluted solution (50 μl) containing the above-mentioned test compound diluted in the buffers and 4 μM buffer solution (25 μl) of a substrate Ac-Asp-Glu-Val-Asp-4-methyl-coumaryl-7-amide (PEPTIDE INSTITUTE, INC.) are dispensed to a light shading type 96 well microplate per well. Thereto is added 25 μl of human recombinant caspase 3 (CALBIOCHEM catalog No. 235417), 500 U/ml, to start the reaction. After incubation at room temperature for 2 hr. the reaction quench buffer (0.1 M monochloroacetic acids 0.03 M sodium acetate, 0.15 M acetic acid (pH 4.3)) is added by 100 μl per well. The fluorescence of 7-amino-4-methylcoumarin (AfC) released by the enzyme reaction is measured using a fluorophotometer (Ex. 355 nm, Em. 460 nm). The enzyme activity is calculated from the analytical curve based on the fluorescence strength of AMC (PEPTIDE INSTITUTE, INC.).

### (3. Assay Method of Inhibitory Activity Against Human Intracellular Caspase-Like Activity)

Similar reaction was carried out using an extract of human culture cell (JOSK-I cell) as an enzyme source, instead of human recombinant caspase 3, and the inhibitory activity against the human intracellular caspase-like activity was confirmed.

JOSK-I cell is cultured in RPMI 1640 medium in a tissue culture flask at 37° C. When the number of the cells exceeds 3×10⁶ cell/ml and apoptosis begins to appear, the culture is centrifuged, washed with ice-cooled phosphate buffer, and suspended in ice-cooled lysis buffer (10 mM Tris (pH 8.13), 5 mM dithiothreitol, 1 mM phenylmethanesulfonyl fluoride, 10 g/ml leupeptin and 10 μg/ml pepstatin). This is frozen and thawed 2 or 3 times in dry ice-acetone and a warm bath (37° C.). This is centrifuged (2000 rpm 15 min, 10000 rpm 10 min) and the supernatant is filtered through a Millipore filter (MilliPore, pore size 0.45 μm) and preserved at −70° C. A thawed solution in a warm bath (37° C.) is added by 25 μl to the above-mentioned reaction mixture to perform the reaction and assay in the same manner as above.

### Example 1

### Activity Inhibitory Action of Cobalt Porphyrin Complex Compound and Cobalt Choline Complex Compound on Human Recombinant Caspase 3

The compounds of the above-mentioned 1. were examined for the activity inhibitory action on human recombinant caspase 3. In this experiment, acetyl-L-aspartyl-L-glutamyl-L-valyl-L-aspart-1-al (DEVD) (PEPTIDE INSTITUTE, INC.) was used as the standard product of caspase 3 inhibitor.

As is clear from the experiment results (FIG. 1), these various cobalt choline complex compounds and cobalt porphyrin complex compounds were found to have a caspase 3 inhibitory activity.

Particularly, as compared to the inhibitory activity of DEVD, a standard product of caspase 3 inhibitor, dicyanocobinamide showed an inhibitory activity against caspase 3, which was far stronger than the inhibitory activity of DEVD.

### Example 2

### Specificity of Inhibitory Activity Against Human Recombinant Caspase 3

As an inhibitor, dicyanocobinamide found to have a strong caspase 3 inhibitory activity in the above-mentioned Example 1 was selected, and the present compound was investigated as to the specificity of the enzyme inhibitory activity.

The above-mentioned compound was assayed for the inhibitory activity using cathepsin E, cathepsin L and trypsin as enzymes other than caspase 3 and by an assay method similar to the above-mentioned Example 1 using phosphate buffer, pH 5.5 (100 mM NaCl, 5-mM DTT, 4 mM EDTA) as a buffer, Z-Arg-Arg-MNA as a substrate for cathepsin H, Z-Phe-Arg-MNA as a substrate for cathepsin L, and Boc-Phe-Ser-Arg-MCA as a substrate for trypsin.

As is clear from the experiment results (FIG. 2), dicyanocobinamide (lower panel of FIG. 2) was confirmed to also have the strongest inhibitory activity against caspase 3, like the standard product DEVD (upper panel of FIG. 2).

### Example 3

### Inhibitory Mode Against Human Recombinant Caspase 3

Using dicyanocobinamide as a caspase 3 inhibitor, experiments similar to the above-mentioned Example 1 were performed with different substrate concentrations (2, 4, 6 and 8 nM). The experiment results are shown in FIG. 3 as Lineweaver-Burk plot. The results suggest that dicyanocobinamide competitively inhibits the substrate for caspase 3

### INDUSTRIAL APPLICABILITY

According to the present invention, a caspase inhibitor comprising a cobalt porphyrin complex compound or cobalt choline complex compound as an active ingredient can be provided. The caspase inhibitor of the present invention is particularly useful for the prophylaxis or treatment of diseases relating to apoptosis.

This application is based on application No. 2004-331331 filed in Japan, the contents of which are incorporated hereinto by reference.

1. A caspase inhibitor comprising a cobalt porphyrin complex compound or a cobalt choline complex compound as an active ingredient.

2. The caspase inhibitor of claim 1, wherein said cobalt porphyrin complex compound or cobalt choline complex compound is a cobyrinic acid derivative.

3. The caspase inhibitor of claim 2, wherein said cobyrinic acid derivative is a cobyrinic acid derivative.

4. The caspase inhibitor of claim 3, wherein said cobyric acid derivative is dicyanocobinamide.

5. The caspase inhibitor of claim 1, wherein said caspase is caspase 3.

6. A pharmaceutical composition for the prophylaxis or treatment of an apoptosis-associated disease, which comprises a cobalt porphyrin complex compound or cobalt choline complex compound in an amount effective for the treatment.

7. The pharmaceutical composition of claim 6, wherein said cobalt porphyrin complex compound or cobalt choline complex compound is a cobyrinic acid derivative.

8. The pharmaceutical composition of claim 7, wherein said cobyrinic acid derivative is a cobyric acid derivative.

9. The pharmaceutical composition of claim 8, wherein said cobyric acid derivative is dicyanocobinamide.

10. The pharmaceutical composition of claim 6, wherein said apoptosis-associated disease is Alzheimer, Parkinson's syndrome or amyotropic lateral sclerosis.

11. A method for inhibiting caspase, comprising applying a cobalt porphyrin complex compound or cobalt choline complex compound.

12. The method of claim 11, wherein said cobalt porphyrin complex compound or cobalt choline complex compound is a cobyrinic acid derivative.

13. The method of claim 12, wherein said cobyrinic acid derivative is a cobyric acid derivative.

14. The method of claim 13, wherein said cobyric acid derivative is dicyanocobinamide.

15. The method of claim 11, wherein said caspase is caspase 3.

16. A method for the prophylaxis or treatment of an apoptosis-associated disease, comprising a step for administering a cobalt porphyrin complex compound or cobalt choline complex compound in an amount effective for the treatment.

17. The method of claim 16, wherein said cobalt porphyrin complex compound or cobalt choline complex compound is a cobyrinic acid derivative.

18. The method of claim 17, wherein said cobyrinic acid derivative is a cobyric acid derivative.

19. The method of claim 18, wherein said cobyrinic acid derivative is dicyanocobinamide.

20. The method of claim 16, wherein said apoptosis-associated disease is Alzheimer, Parkinson's syndrome or amyotropic lateral sclerosis.

21. Use of a cobalt porphyrin complex compound or cobalt choline complex compound for the production of a caspase inhibitor.

22. The use of claim 21, wherein said cobalt porphyrin complex compound or cobalt choline complex compound is a cobyrinic acid derivative.

23. The use of claim 22, wherein said cobyrinic acid derivative is a cobyric acid derivative.

24. The use of claim 23, wherein said cobyric acid derivative is dicyanocobinamide.

25. The use of claim 21, wherein said caspase is caspase 3.

26. Use of a cobalt porphyrin complex compound or cobalt choline complex compound for the production of a pharmaceutical agent for the prophylaxis or treatment of an apoptosis-associated disease.

27. The use of claim 26, wherein said cobalt porphyrin complex compound or cobalt choline complex compound is a cobyrinic acid derivative.

28. The use of claim 2, wherein said cobyrinic acid derivative is a cobyric acid derivative.

29. The use of claim 28, wherein said cobyric acid derivative is dicyanocobinamide.

30. The use of claim 26, wherein said apoptosis-associated disease is Alzheimer, Parkinson's syndrome or amyotropic lateral sclerosis.

31. A commercial package comprising the caspase inhibitor of claim 1 and a written matter associated therewith, the written matter stating that the caspase inhibitor can or should be used for the prophylaxis or treatment of an apoptosis-associated disease.

32. The commercial package of claim 31, wherein said apoptosis-associated disease is Alzheimer, Parkinson's syndrome or amyotropic lateral sclerosis.
